(19) United States
(12) Patent Application Publication
(10) Pub. No.: US 2005/0245517 A1
(43) Pub. Date: Nov. 3, 2005

(54) 2-pyridinyl[7-(substituted-pyridin-4-yl) pyrazolo[1,5-a]pyrimidin-3-yl]methanones

(76) Inventors: Phil Skolnick, Edgewater, NJ (US); Joseph William Epstein, Monroe, NY (US)

Correspondence Address: GRAYBEAL JACKSON HALEY LLP;Suite. 350, 155-108th Avenue N.E., Bellevue, WA, 98004-5973, US

(21) Appl. No.: 11/118,111

(22) Filed: Apr. 29, 2005

Related U.S. Application Data
(60) Provisional application No. 60/566,532, filed on Apr. 29, 2004.

(51) Int. Cl. A61K 31/5377; A61K 31/519; C07D 487/04

(52) U.S. Cl. 514/234.5; 514/259.3; 544/114; 544/281

(57) ABSTRACT

The present invention provides novel 2-pyridinyl[7(pyridin-4-yl)pyrazolo[1,5-a]pyrimidin-3-yl]methanones with at least one substituent on both the 2- and 4-pyridinyl ring having the chemical structure of formula I: 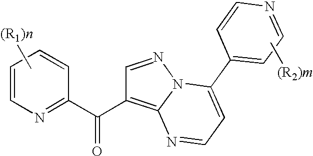
The invention further provides compositions and methods employing the novel 2-pyridinyl[7-(pyridin-4-yl)pyrazolo[1,5-a]pyrimidin-3-yl]methanones of formula I to modulate GABA and GABA_{A} receptor physiology to elicit therapeutic responses in mammalian subjects to alleviate neurological or psychiatric disorders, including stroke, head trauma, epilepsy, pain, migraine, mood disorders, anxiety, post traumatic stress disorder, obsessive compulsive disorders, mania, bipolar disorders, schizophrenia, seizures, convulsions, tinnitus, neurodegenerative disorders including Alzheimer's disease, amyotrophic lateral sclerosis and Parkinson's disease, Huntington's chorea, depression, bipolar disorders, mania, trigeminal and other neuralgia, neuropathic pain, hypertension, cerebral ischemia, cardiac arrhythmia, myotonia, substance abuse, myoclonus, essential tremor, dyskinesia and other movement disorders, neonatal cerebral hemorrhage, and spasticity, as well as other psychiatric and neurological disorders mediated by GABA and/or GABA_{A} receptors.

### RELATED APPLICATIONS

This application claims the benefit of U.S. Provisional Application No. 60/566,532, filed Apr. 29, 2004, which is incorporated herein by reference.

### TECHNICAL FIELD

The present invention relates to novel 2-pyridinyl[7-(pyridin-4-yl)pyrazolo[1,5-a]pyrimidin-3-yl]methanones having substituents on both the 2- and 4-pyridinyl rings and pharmaceutical compositions containing the same.

### BACKGROUND OF THE INVENTION

γ-Aminobutyric acid (GABA) (C₄H₉NO₂) is the most common inhibitory neurotransmitter in the mammalian brain and is estimated to be present at about one third of all synapses. When GABA binds to a GABA receptor, it affects the ability of neurons expressing the receptors to conduct neural impulses. In the adult mammalian nervous system, GABA typically inhibits neuron firing (depolarization). Neurons in the brain express three main types of GABA receptors, GABA_{A}, GABA_{B}, and GABA_{C}. GABA_{A} receptors function as ligand-gated ion channels to mediate fast inhibitory synaptic transmissions that regulate neuronal excitability involved in such responses as seizure threshold, skeletal muscle tone, and emotional status. GABA_{A} receptors are targets of many sedating drugs, such as benzodiazepines, barbiturates, neurosteroids, and ethanol.

The intrinsic inhibitory signal of GABA is transduced principally by GABA_{A} receptors. GABA_{A} receptors are pentameric, ligand-gated chloride ion (Cl⁻) channels belonging to a superfamily of ligand-gated ionotropic receptors that includes the nicotinic acetylcholine receptor. GABA_{A} receptors are very heterogeneous, with at least 16 different subunits producing potentially thousands of different receptor types. The distinct protein subunits fall into homologous families denoted as α₁₋₆, β₁₋₄, γ₁₋₃, δ, ε, θ, and ρ₁₋₃ (Barnard, et al., Pharmacol. Rev. 50:291-313, 1998). The major isoform of the GABA_{A} receptor in the adult mammalian brain consists of 2α₁, 2β₂, and a single γ₂ subunit, although receptors containing different subunit combinations are found in different brain regions at different times in development, and likely serve some unique functions (Wisden et al., J. Neurosci. 12:1040-1062, 1992).

GABA_{A} receptor subunits aggregate into complexes that form chloride ion selective channels and contain sites that bind GABA along with a variety of pharmacologically active substances. When GABA binds to this receptor, the anion channel is activated, causing it to open and allowing chloride ions (Cl⁻) to enter the neuron. This influx of Cl⁻ ions hyperpolarizes the neuron, making it less excitable. The resultant decrease in neuronal activity following activation of the GABA_{A} receptor complex can rapidly alter brain function, to such an extent that consciousness and motor control may be impaired.

The numerous possible combinations of GABA_{A} receptor subunits and the widespread distribution of these receptors in the nervous system likely contributes to the diverse and variable physiological functions of GABA_{A} receptors, which have been implicated in many neurological and psychiatric disorders, and related conditions, including: stroke, head trauma, epilepsy, pain, migraine, mood disorders, anxiety, post traumatic stress disorder, obsessive compulsive disorders, schizophrenia, seizures, convulsions, tinnitus, neurodegenerative disorders including Alzheimer's disease, amyotrophic lateral sclerosis, Huntington's Chorea, Parkinson's disease, depression, bipolar disorders, mania, trigeminal and other neuralgia, neuropathic pain, hypertension, cerebral ischemia, cardiac arrhythmia, myotonia, substance abuse, myoclonus, essential tremor, dyskinesia and other movement disorders, neonatal cerebral hemorrhage, and spasticity. GABA_{A} receptors are also believed to play a role in cognition, consciousness, and sleep.

Currently-available drugs for modulating GABA_{A} receptor activity include barbiturates, such as pentobarbital and secobarbital, and benzodiazepines such as diazepam, chlordiazepoxide and midazolam. Barbiturates can directly activate GABA_{A} receptors, significantly increasing Cl⁻ currents in the absence of further intervention by GABA itself and can also indirectly augment GABAergic neural transmission. In contrast, benzodiazepines act as indirect allosteric modulators, and are largely incapable of increasing Cl⁻ currents in the absence of GABA, but enhance GABA-activated increases in Cl⁻ conductance. This latter property is thought to be responsible for the usefulness of benzodiazepines for treating a number of disorders, including generalized anxiety disorder, panic disorder, seizures, movement disorders, epilepsy, psychosis, mood disorders, and muscle spasms, as well as the relative safety of benzodiazepines compared to barbiturates.

Both barbiturates and benzodiazepines are addictive and can cause drowsiness, poor concentration, ataxia, dysarthria, motor incoordination, diplopia, muscle weakness, vertigo and mental confusion. These side effects can interfere with an individual's ability to perform daily routines such as driving, operating heavy machinery or performing other complex motor tasks while under therapy, making barbiturates and benzodiazepines less than optimal for treating chronic disorders involving GABA and GABA_{A} receptors.

GABA_{A} receptors are implicated as targets for therapeutic intervention in a myriad of neurological and psychiatric disorders. Adverse side effects, including addictive properties exhibited by currently available GABA and GABA receptor modulating drugs, make these drugs unsuitable in many therapeutic contexts. Accordingly, there remains an important, unmet need in the art for alternative compositions, methods and tools that will be useful in broad clinical applications to modulate the function and activity of GABA and GABA receptors.

It is therefore an object of the present invention to provide novel and improved compositions and methods for modulating the function and activity of GABA and/or GABA_{A} receptors in mammalian subjects, including humans.

It is another object of the present invention to provide novel and improved compositions and methods for treating psychiatric and neurological disorders involving a deficit in GABAergic neural transmission, including compositions and methods that are effective in the treatment of anxiety disorders, seizure disorders, tinnitus, affective disorders, pain, muscle spasms, schizophrenia, and cognitive disorders in mammalian subjects requiring such treatment.

### SUMMARY OF EXEMPLARY EMBODIMENTS OF THE INVENTION

The invention achieves these objects and satisfies additional objects and advantages by providing novel 2-pyridinyl[7-(pyridin-4-yl)pyrazolo[1,5-a]pyrimidin-3-yl]methanones having at least one substituent on both the 2- and 4-pyridinyl rings of formula I: 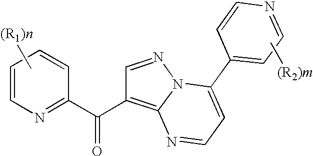
Wherein, each R₁ and R₂ can independently be a halogen, hydroxy, alkyl, alkoxy, nitro, amino, trifluoromethyl, cycloalkyl, (cycloalkyl)alkyl, alkanoyl, alkanoyloxy, aryl, aroyl, aralkyl, nitrile, pyrrolidine-1-yl, morpholino, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, alkanoylamino, carbamoyl, carbamyl, carbonylamino, alkylsulfonylamino, or heterocyclo groups. Furthermore, each of the R₁ and R₂ groups may be optionally substituted and when n or m is greater than 1, two R₁ or R₂ groups, for example adjacent R₁ or R₂ groups, may be fused to form a five-or six-membered ring with the two carbons on the pyridinyl ring to which they are attached. Furthermore, when n or m is greater than one, each R₁ or R₂ group may be selected independently. Thus, when more than one R₁ group is present, each R₁ group may be selected from any of the stated groups so as to be the same or different. Similarly, when more than one R₂ group is present, each R₂ group may be selected from any of the stated groups so as to be the same or different.

Within exemplary embodiments, the invention provides novel 2-pyridinyl[7-(pyridin-4-yl)pyrazolo[1,5-a]pyrimidin-3-yl]methanones having at least one substituent on both the 2- and 4-pyridinyl rings, which are capable of modulating GABA or GABA_{A} receptor function or activity, including activity or function mediated by GABA_{A} receptors.

Useful forms of novel 2-pyridinyl[7-(pyridin-4-yl)pyrazolo[1,5-a]pyrimidin-3-yl]methanones of the invention, having at least one substituent on both the 2- and 4-pyridinyl rings, include various pharmaceutically acceptable salts, polymorphs, solvates, hydrates and/or prodrugs of these substituted compounds, as well as combinations thereof. In exemplary embodiments, the compositions and methods of the invention may employ 2-pyridinyl[7-(pyridin-4-yl)pyrazolo[1,5-a]pyrimidin-3-yl]methanones having at least one substituent on both the 2- and 4-pyridinyl rings as GABA or GABA_{A} receptor modulators, capable of detectably modulating one or more activity(ies) or function(s) of GABA or of a GABA_{A} receptor.

Mammalian subjects amenable for treatment using the compositions and methods of the invention include, but are not limited to, human and other mammalian subjects suffering from a psychiatric or neurological disorder mediated, at least in part, by a dysfunction or imbalance in GABA or GABA_{A} receptor physiology. The compounds and methods of the invention can be effectively employed to alleviate or prevent one or more symptoms of a psychiatric or neurological disorder, or a related condition, including stroke, head trauma, epilepsy, pain, migraine, mood disorders, anxiety, post traumatic stress disorder, obsessive compulsive disorders, bipolar disorders, psychotic disorders including schizophrenia, seizures, convulsions, tinnitus, neurodegenerative disorders including Alzheimer's disease, amyotrophic lateral sclerosis, Parkinson's disease, Huntington's chorea, depression, bipolar disorders, mania, trigeminal and other neuralgia, neuropathic pain, hypertension, cerebral ischemia, cardiac arrhythmia, myotonia, substance abuse, myoclonus, essential tremor, dyskinesia and other movement disorders, neonatal cerebral hemorrhage, and spasticity.

These and other subjects are effectively treated according to the invention by administering to the subject an effective amount of a 2-pyridinyl[7-(pyridin-4-yl)pyrazolo[1,5-a]pyrimidin-3-yl]methanone compound having at least one substituent on both the 2- and 4-pyridinyl ring, which compound is effective to modulate a function or activity of GABA, or of a GABA_{A} receptor. Often, the novel compounds of the invention will modulate GABA binding to a GABA_{A} receptor. The active compounds of the invention are provided in a variety of forms, including pharmaceutically acceptable salts, polymorphs, solvates, hydrates and/or prodrugs of a 2-pyridinyl[7(pyridin-4-yl)pyrazolo[1,5-a]pyrimidin-3-yl]methanone compound having at least one substituent on both the 2- and 4-pyridinyl rings.

Within additional aspects of the invention, combinatorial formulations and methods are provided comprising an effective amount of 2-pyridinyl[7-(pyridin-4-yl)pyrazolo[1,5-a]pyrimidin-3-yl]methanone compounds having at least one substituent on both the 2- and 4-pyridinyl rings and one or more additional active agents combinatorially formulated or coordinately administered with the substituted 2-pyridinyl[7-(pyridin-4-yl)pyrazolo[1,5-a]pyrimidin-3-yl]methanone compound, to elicit a GABA or GABA_{A} receptor modulating response in a mammalian subject. Exemplary combinatorial formulations and coordinate treatment methods in this context employ 2-pyridinyl[7-(pyridin-4-yl)pyrazolo[1,5-a]pyrimidin-3-yl]methanones having at least one substituent on both the 2- and 4-pyridinyl rings in combination with one or more additional GABA or GABA_{A} receptor modulators, including adjunctive agents selected from analgesics, anxiolytics, antidepressants, anticonvulsants, nootropics, anesthetics, hypnotics and muscle relaxants.

The forgoing objects and additional objects, features, aspects and advantages of the instant invention will become apparent from the following detailed description.

### DETAILED DESCRIPTION OF EXEMPLARY EMBODIMENTS OF THE INVENTION

The instant invention provides novel 2-pyridinyl[7-(pyridin-4-yl)pyrazolo[1,5-a]pyrimidin-3-yl]methanones having at least one substituent on both the 2- and 4-pyridinyl rings. Also provided are compositions and methods for using these novel methanones to treat psychiatric and neurological disorders in mammals involving GABA and GABA_{A} receptors. In various embodiments, the methods and compositions of the invention are effective as anxiolytic, antidepressant, anticonvulsant, nootropic, anesthetic, hypnotic, and/or muscle relaxant agents in therapies and formulations of the invention.

Formulations and methods provided herein employ 2-pyridinyl[7-(pyridin-4-yl)pyrazolo[1,5-a]pyrimidin-3-yl]methanones having at least one substituent on both the 2- and 4-pyridinyl rings for treating psychiatric and neurological disorders in mammalian subjects, typically disorders mediated by a dysfunction or imbalance in endogenous GABA and/or GABA_{A} receptor physiology in the subject. Within these formulations and methods, the 2-pyridinyl[7-(pyridin-4-yl)pyrazolo[1,5-a]pyrimidin-3-yl]methanones having at least one substituent on both the 2- and 4-pyridinyl rings can be provided in any of a variety of forms, including any pharmaceutically acceptable salt, solvate, hydrate, polymorph, or prodrug of the substituted methanone compound, and combinations thereof.

The novel compounds of the invention, comprising 2-pyridinyl[7-(pyridin-4-yl)pyrazolo[1,5-a]pyrimidin-3-yl]methanones having at least one substituent on both the 2- and 4-pyridinyl rings, will typically possess GABA or GABA_{A} receptor modulatory activity. In this context, GABA and GABA_{A} receptor modulatory agents of the invention will frequently bind or interact with sites on a GABA_{A} receptor complex, such as the benzodiazepine receptor, and can have either an enhancing effect on the action of GABA, an attenuating effect on the action of GABA, or a dual activity blockade effect capable of modulating both enhancing and attenuating activities or functions of GABA and/or GABA_{A} receptors. Certain compounds of the invention will be agonists, or enhancing agents, and will often possess activity to mediate muscle relaxant, hypnotic, sedative, anxiolytic, and/or anticonvulsant effects in the subject. Other compounds of the invention will be inverse agonists, or attenuating agents, capable of producing pro-convulsive, anti-inebriant or anxiogenic effects. Still other compounds of the invention will be partial agonists, mediating anxiolytic effects, with or without reduced muscle relaxant, hypnotic and sedative effects. In more detailed embodiments, partial inverse agonist compounds are provided which will be useful as cognition enhancers.

A broad range of mammalian subjects, including human subjects, are amenable for treatment using the formulations and methods of the invention. These subjects include, but are not limited to, human and other mammalian subjects suffering from stroke, head trauma, epilepsy, pain, migraine, mood disorders, anxiety, post traumatic stress disorder, obsessive compulsive disorders, mania, bipolar disorders, schizophrenia, seizures, convulsions, tinnitus, neurodegenerative disorders including Alzheimer's disease, amyotrophic lateral sclerosis, Parkinson's disease, Huntington's chorea, depression, bipolar disorders, mania, trigeminal and other neuralgia, neuropathic pain, hypertension, cerebral ischemia, cardiac arrhythmia, myotonia, substance abuse, myoclonus, essential tremor, dyskinesia and other movement disorders, neonatal cerebral hemorrhage, and spasticity, among various other psychiatric and neurological disorders associated with impaired function or activity of GABA and/or GABA_{A} receptors.

Within certain methods and composition of the invention, 2-pyridinyl[7-(pyridin-4-yl)pyrazolo[1,5-a]pyrimidin-3-yl]methanone compounds having at least one substituent on both the 2- and 4-pyridinyl rings are effectively formulated and administered to treat or prevent a neurological or psychiatric disorder in a mammalian subject. Most often, the therapeutic or prophylactic effect of these compounds involves direct modulation of an activity or function of GABA, or of a GABA_{A} receptor, by the administered compound.

The 2-pyridinyl[7-(substituted-pyridin-4-yl)pyrazolo[1,5-a]pyrimidin-3-yl]methanones with substituents on the 2- and 4-pyridinyl rings, provided in accordance with the present invention, include derivatives of the reported anxiolytic agent ocinaplon, (2-pyridinyl)-[7-(4-pyridinyl)pyrazolo[1,5-a]pyrimidin-3-yl]methanone, which is represented 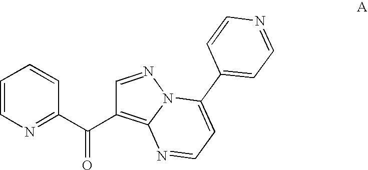
by the structural formula A and is described in U.S. Pat. No. 4,521,422 to Dusza et al. (“Dusza”), issued Jun. 4, 1985:

The Dusza patent contemplates a genus encompassing in excess of ten million compounds, a number of sub-genera encompassing some eight thousand compounds, primarily phenyl[7-(4-pyridinyl)pyrazolo[1,5-a]pyrimidin-3-yl]methanones and 2-furanyl[7-(4-pyridinyl)pyrazolo[1,5-a]pyrimidin-3-yl]methanones, and exemplifies 222 specific compounds, none of which have substituents on the 2- and 4-pyridinyl rings.

The novel compounds of the present invention are represented by structural formula I: 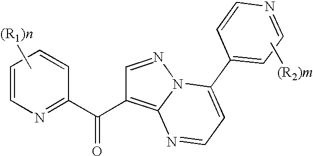
In formula I, R₁ and/or R₂ can each independently be a halogen, hydroxy, alkyl, alkoxy, nitro, amino, trifluoromethyl, cycloalkyl, (cycloalkyl)alkyl, alkanoyl, alkanoyloxy, aryl, aroyl, aralkyl, nitrile, pyrrolidine-1-yl, morpholino, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, alkanoylamino, carbamoyl, carbamyl, carbonylamino, alkylsulfonylamino, and heterocyclo groups. In one embodiment, each of the R₁ and/or R₂ groups may be optionally substituted as described herein. In another embodiment, two R₁ and/or R₂ groups may be fused to form a five-or six-membered ring with the two carbons on the pyridinyl ring to which they are attached. When n or m is greater than one, each R group may be selected independently. Thus, when more than one R₁ group is present, each R₁ group may be selected from any of the stated groups so as to be the same or different. Similarly, when more than one R₂ group is present, each R₂ group may be selected from any of the stated groups so as to be the same or different.

The term “halogen” as used herein refers to bromine, chlorine, fluorine or iodine. In one embodiment, the halogen is chlorine. In another embodiment, the halogen is bromine.

The term “hydroxy” as used herein refers to —OH or —O⁻.

The term “alkyl” as used herein refers to straight- or branched-chain aliphatic groups containing 1-20 carbon atoms, preferably 1-7 carbon atoms and most preferably 1-4 carbon atoms. This definition applies as well to the alkyl portion of alkoxy, alkanoyl and aralkyl groups. In one embodiment, the alkyl is a methyl group.

The term “alkoxy” includes substituted and unsubstituted alkyl, alkenyl, and alkynyl groups covalently linked to an oxygen atom. In one embodiment, the alkoxy group contains 1 to 4 carbon atoms. Embodiments of alkoxy groups include, but are not limited to, methoxy, ethoxy, isopropyloxy, propoxy, butoxy, and pentoxy groups. Embodiments of substituted alkoxy groups include halogenated alkoxy groups. In a further embodiment, the alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Exemplary halogen substituted alkoxy groups include, but are not limited to, fluoromethoxy, difluoromethoxy, trifluoromethoxy, chloromethoxy, dichloromethoxy, and trichloromethoxy.

The term “nitro”, as used herein alone or in combination refers to a —NO₂ group.

The term “amino” as used herein refers to the group —NRR′, where R and R′ may independently be hydrogen, alkyl, aryl, alkoxy, or heteroaryl. The term “aminoalkyl” as used herein represents a more detailed selection as compared to “amino” and refers to the group —NRR′, where R and R′ may independently be hydrogen or (C₁-C₄)alkyl.

The term “trifluoromethyl” as used herein refers to —CF₃.

The term “trifluoromethoxy” as used herein refers to —OCF₃

The term “cycloalkyl” as used herein refers to a saturated cyclic hydrocarbon ring system containing from 3 to 7 carbon atoms that may be optionally substituted. Exemplary embodiments include, but are not limited to, cyclopropyl, cyclobutyl, cyclopentyl and cyclohexyl. In certain embodiments, the cycloalkyl group is cyclopropyl. In another embodiment, the (cycloalkyl)alkyl groups contain from 3 to 7 carbon atoms in the cyclic portion and 1 to 4 carbon atoms in the alkyl portion. In certain embodiments, the (cycloalkyl)alkyl group is cyclopropylmethyl. The alkyl groups are optionally substituted with from one to three substituents selected from the group consisting of halogen, hydroxy and amino.

The terms “alkanoyl” and “alkanoyloxy” as used herein refer, respectively, to —C(O)-alkyl groups and —O—C(O)-alkyl groups, each optionally containing 2-5 carbon atoms. Specific embodiments of alkanoyl and alkanoyloxy groups are acetyl and acetoxy, respectively.

The term “aryl” as used herein refers to monocyclic or bicyclic aromatic hydrocarbon groups having from 6 to 12 carbon atoms in the ring portion, for example, phenyl, naphthyl, biphenyl and diphenyl groups, each of which may be substituted with, for example, one to four substituents such as alkyl; substituted alkyl as defined above, halogen, trifluoromethyl, trifluoromethoxy, hydroxy, alkoxy, cycloalkyloxy, alkanoyl, alkanoyloxy, amino, alkylamino, dialkylamino, nitro, cyano, carboxy, carboxyalkyl, carbamyl, carbamoyl and aryloxy. Specific embodiments of aryl groups in accordance with the present invention include phenyl, substituted phenyl, naphthyl, biphenyl, and diphenyl.

The term “aroyl,” as used alone or in combination herein, refers to an aryl radical derived from an aromatic carboxylic acid, such as optionally substituted benzoic or naphthoic acids.

The term “aralkyl” as used herein refers to an aryl group bonded to the 2-pyridinyl ring and/or the 4-pyridinyl ring through an alkyl group, preferably one containing 1-4 carbon atoms. A preferred aralkyl group is benzyl.

The term “nitrile” or “cyano” as used herein refers to the group —CN.

The term “pyrrolidine-1-yl” as used herein refers to the structure:

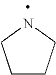

The term “morpholino” as used herein refers to the structure:

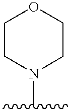

The term “dialkylamino” refers to an amino group having two attached alkyl groups that can be the same or different.

The term “alkenyl” refers to a straight or branched alkenyl group of 2 to 10 carbon atoms having 1 to 3 double bonds. Preferred embodiments include ethenyl, 1-propenyl, 2-propenyl, 1-methylethenyl, 1-butenyl, 2-butenyl, 3-butenyl, 2-methyl-2-propenyl, 1-pentenyl, 2-pentenyl, 4-pentenyl, 3-methyl-2-butenyl, 1-hexenyl, 2-hexenyl, 1-heptenyl, 2-heptenyl, 1-octenyl, 2-octenyl, 1,3-octadienyl, 2-nonenyl, 1,3-nonadienyl, 2-decenyl, etc.

The term “alkynyl” as used herein refers to a straight or branched alkynyl group of 2 to 10 carbon atoms having 1 to 3 triple bonds. Exemplary alkynyls include, but are not limited to, ethynyl, 1-propynyl, 2-propynyl, 1-butynyl, 2-butynyl, 3-butynyl, 1-pentynyl, 2-pentynyl, 4-pentynyl, 1-octynyl, 6-methyl-1-heptynyl, and 2-decynyl.

The term “hydroxyalkyl” alone or in combination, refers to an alkyl group as previously defined, wherein one or several hydrogen atoms, preferably one hydrogen atom has been replaced by a hydroxyl group. Examples include hydroxymethyl, hydroxyethyl and 2-hydroxyethyl.

The term “aminoalkyl” as used herein refers to the group —NRR′, where R and R′ may independently be hydrogen or (C₁-C₄)alkyl.

The term “alkylaminoalkyl” refers to an alkylamino group linked via an alkyl group (i.e., a group having the general structure --alkyl-NH-alkyl or --alkyl-N(alkyl)(alkyl)). Such groups include, but are not limited to, mono- and di-(C₁-C₈ alkyl)aminoC₁-C₈ alkyl, in which each alkyl may be the same or different.

The term “dialkylaminoalkyl” refers to alkylamino groups attached to an alkyl group. Examples include, but are not limited to, N,N-dimethylaminomethyl, N,N-dimethylaminoethyl N,N-dimethylaminopropyl, and the like. The term dialkylaminoalkyl also includes groups where the bridging alkyl moiety is optionally substituted.

The term “haloalkyl” refers to an alkyl group substituted with one or more halo groups, for example chloromethyl, 2-bromoethyl, 3-iodopropyl, trifluoromethyl, perfluoropropyl, 8-chlorononyl and the like.

The term “carboxyalkyl” as used herein refers to the substituent —R′—COOH wherein R′ is alkylene; and carbalkoxyalkyl refers to —R′—COOR wherein R′ and R are alkylene and alkyl respectively. In certain embodiments, alkyl refers to a saturated straight- or branched-chain hydrocarbyl radical of 1-6 carbon atoms such as methyl, ethyl, n-propyl, isopropyl, n-butyl, t-butyl, n-pentyl, 2-methylpentyl, n-hexyl, and so forth. Alkylene is the same as alkyl except that the group is divalent.

The term “alkoxyalkyl” refers to a alkylene group substituted with an alkoxy group. For example, methoxyethyl [CH₃OCH₂CH₂—] and ethoxymethyl [CH₃CH₂OCH₂—] are both C₃ alkoxyalkyl groups.

The term “carboxy”, as used herein, represents a group of the formula—COOH.

The term “alkanoylamino” refers to alkyl, alkenyl or alkynyl groups containing the group —C(O)— followed by —N(H)—, for example acetylamino, propanoylamino and butanoylamino and the like.

The term “carbonylamino” refers to the group —NR—CO—CH₂—R′, where R and R′ may be independently selected from hydrogen or (C₁-C₄)alkyl.

The term “carbamoyl” as used herein refers to —O—C(O)NH₂.

The term “carbamyl” as used herein refers to a functional group in which a nitrogen atom is directly bonded to a carbonyl, i.e., as in —NRC(═O)R′ or —C(═O)NRR′, wherein R and R′ can be hydrogen, alkyl, substituted alkyl, alkenyl, substituted alkenyl, alkoxy, cycloalkyl, aryl, heterocyclo, or heteroaryl.

The term “alkylsulfonylamino” refers to refers to the group —NHS(O)₂Rₐ wherein Rₐ is an alkyl as defined above.

The term “heterocyclo” refers to an optionally substituted, unsaturated, partially saturated, or fully saturated, aromatic or nonaromatic cyclic group that is a 4 to 7 membered monocyclic, or 7 to 11 membered bicyclic ring system that has at least one heteroatom in at least one carbon atom-containing ring. The substituents on the heterocyclo rings may be selected from those given above for the aryl groups. Each ring of the heterocyclo group containing a heteroatom may have 1, 2 or 3 heteroatoms selected from nitrogen atoms, oxygen atoms and sulfur atoms. Plural heteroatoms in a given heterocyclo ring may be the same or different. The heterocyclo group may be attached to the 2- and/or 4-pyridinyl ring at any heteroatom or carbon atom. In one embodiment, the heterocyclo group attaches to the 2-pyrindyl ring forming a 2-isoquinolinyl moiety. In another embodiment, the heterocyclo group attaches to the 4-pyridinyl ring forming a 7-quinolin-4-yl moiety. In a further embodiment, the heterocyclo group may attach to the 2-pyrindyl ring forming a 2-isoquinolinyl moiety and the same or a different heterocyclo may attach to the 4-pyridinyl ring forming a 7-quinolin-4-yl moiety. In yet another embodiment, two R groups form a fused ring with the carbons at position 2 and 3 of the pyridinyl ring, forming a 7-quinolin-4-yl moiety.

Exemplary monocyclic heterocyclo groups include pyrrolidinyl, pyrrolyl, indolyl, pyrazolyl, imidazolyl, oxazolyl, isoxazolyl, thiazolyl, furyl, tetrahydrofuryl, thienyl, piperidinyl, piperazinyl, azepinyl, pyrimidinyl, pyridazinyl, tetrahydropyranyl, morpholinyl, dioxanyl, triazinyl and triazolyl. Preferred bicyclic heterocyclo groups include benzothiazolyl, benzoxazolyl, benzothienyl, quinolinyl, tetrahydroisoquinolinyl, benzimidazolyl, benzofuryl, indazolyl, benzisothiazolyl, isoindolinyl and tetrahydroquinolinyl. In more detailed embodiments heterocyclo groups may include indolyl, imidazolyl, furyl, thienyl, thiazolyl, pyrrolidyl, pyridyl and pyrimidyl.

In exemplary embodiments of the invention, substituents on the 4-pyrindyl ring of the compounds of formula I are on the 2 position, the 2 and 6 positions, the 2 and 5 positions, the 3 position and the 3 and 5 positions. In other exemplary embodiments of the invention, substituents on the 2-pyrindyl ring of the compounds of formula I are on the 3, 4, and 5 positions.

All value ranges expressed herein, for example those given for n or m, are inclusive over the indicated range. Thus, a range of n or m between 0 to 4 will be understood to include the values of 1, 2, 3, and 4. In exemplary embodiments, n and m are integers greater than 0, and n and m are independent. For example, when n is 2, m can be 2 or any other integer. In another embodiment, both n and m are 1. In a further embodiment, n and m are between 0 and 4.

In exemplary embodiments of the invention, the compounds of formula I may include, but are not limited to, (6-methyl-2-pyridinyl)-[7-(2-chloropyridin-4-yl)pyrazolo[1,5-a]pyrimidin-3-yl]methanone; (6-methoxy-2-pyridinyl)-[7-(2-chloropyridin-4-yl)pyrazolo[1,5-a]pyrimidin-3-yl]methanone; (6-methoxy-2-pyridinyl)-[7-(2-bromopyridin-4-yl)pyrazolo[1,5-a]pyrimidin-3-yl]methanone; and (6-methyl-2-pyridinyl)-[7-(2-bromopyridin-4-yl)pyrazolo[1,5-a]pyrimidin-3-yl]methanone.

While the novel 2- and 4-pyridinyl substituted 2-pyridinyl[7-(pyridin-4-yl)pyrazolo[1,5-a]pyrimidin-3-yl]methanone compounds of the present invention may be generated by any methods known to those skilled in the art, they may also be generated, for example, according to General Synthetic Scheme 1 described herein, below. This synthetic scheme is provided for illustrative purposes only, and it is understood that abbreviated, alternate, and modified schemes, e.g., emcompassing essential elements of this scheme, or their equivalents, are also contemplated within the scope of the invention. For example, in one embodiment of the present invention, the novel compounds as described herein may be prepared according to General Synthetic Scheme 1, as follows:

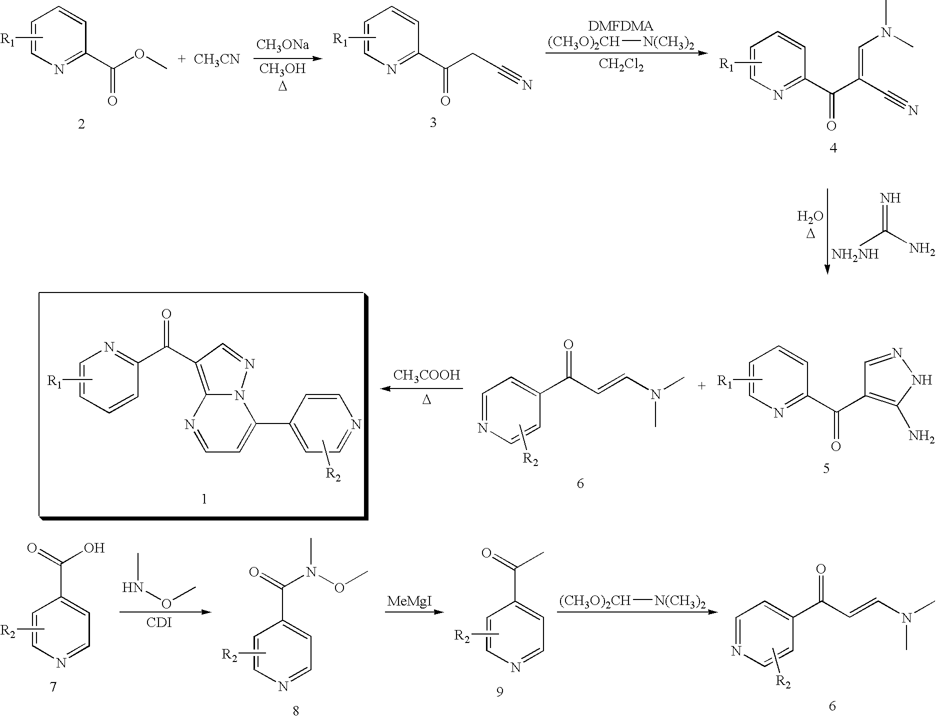

General Synthetic Scheme 1 may be used to generate various compounds of the present invention including, but not limited to, (6-methyl-2-pyridinyl)-[7-(2-chloropyridin-4-yl)pyrazolo[1,5-a]pyrimidin-3-yl]methanone; (6-methoxy-2-pyridinyl)-[7-(2-chloropyridin-4-yl)pyrazolo[1,5-a]pyrimidin-3-yl]methanone; (6-methoxy-2-pyridinyl)-[7-(2-bromopyridin-4-yl)pyrazolo[1,5-a]pyrimidin-3-yl]methanone; and (6-methyl-2-pyridinyl)-[7-(2-bromopyridin-4-yl)pyrazolo[1,5-a]pyrimidin-3-yl]methanone.

The capacity of compounds of formula I to bind to GABA_{A} receptor complexes in the brain was determined through radioligand binding assays, as described in Example 12, below. Competition binding assays were performed using the selective radioligand [³H]Ro 15-1788, which binds to the allosteric modulatory site on GABA_{A} receptors used by compounds such as benzodiazepines to modulate receptor activity. Agents with affinity for the GABA_{A} receptor inhibit or reduce the amount of radioligand bound in this assay. The results of this assay for exemplary compounds of the present invention are shown in Table 1.

TABLE 1
The effect of substitutions on the pyridine-2-yl and 4-yl ring of 2-pyridinyl[7-(pyridin-4-yl)pyrazolo[1,5-
a]pyrimidin-3-yl]methanones on compound affinity for the benzodiazepine receptor

| Compounds of Formula | | | |
|---|---|---|---|
| I, with substituents on | | | “GABA Shift” Ratio |
| the 2 and 4-pyridinyl | | | IC₅₀, μM (−GABA)/ |
| ring | IC₅₀, μM (−GABA) | IC₅₀, μM (+GABA) | IC₅₀, μM (+GABA) |
| 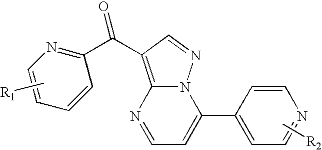 | | | |
| Compound #1 | 54.5 ± 4.7 | 45.7 ± 4.4 | 1.19 |
| R₁ = 6-Me, R₂ = 2-Cl | | | |
| Compound #2 | 164 ± 13 | 124 ± 26 | 1.32 |
| R₁ = 6-Me, R₂ = 2-Br | | | |
| Compound #3 | 20.9 ± 3.5 | 16.9 ± 1.9 | 1.24 |
| R₁ = 6-Me, R₂ = 2-COPh | | | |
| Compound #4 | 0.62 ± 0.20 | 0.35 ± 0.08 | 1.77 |
| R₁ = 5-Me, R₂ = 2-Cl | | | |
| Compound #5 | 0.35 ± 0.04 | 0.28 ± 0.04 | 1.25 |
| R₁ = 5-Me, R₂ = 2-Br | | | |
| Compound #6 | 63.7 ± 8.2 | 52.1 ± 8.8 | 1.22 |
| R₁ = 5-Me, R₂ = 2-COPh | | | |

IC₅₀: Concentration of compound required to inhibit [3H]Ro 15-1788 binding to the benzodiazepine receptor by 50%.

The results presented in Table 1 demonstrate that a diverse assemblage of exemplary compounds of formula I exhibit specific binding to the GABA_{A} receptor, as demonstrated, for example, by the compounds' ability to inhibit [³H]Ro 15-1788 binding to the receptor preparation with an IC₅₀ of less than 10 μM.

The compositions and methods of the instant invention represented by formula I are effective for treating or preventing psychiatric and neurological disorders in mammals. In particular, the compositions and methods of the invention can be administered to mammalian subjects to measurably alleviate or prevent one or more symptoms of a psychiatric or neurological disorder, or a related condition, including, but not limited to, the symptoms of stroke, head trauma, epilepsy, pain, migraine, mood disorders, anxiety, post traumatic stress disorder, obsessive compulsive disorders, bipolar disorders, schizophrenia, seizures, convulsions, tinnitus, neurodegenerative disorders (including Alzheimer's disease, Huntington's chorea, amyotrophic lateral sclerosis and Parkinson's disease), bipolar disorders, mania, depression, trigeminal and other neuralgia, neuropathic pain, hypertension, cerebral ischemia, cardiac arrhythmia, myotonia, substance abuse, myoclonus, essential tremor, dyskinesia and other movement disorders (including, but not limited to, Wilson's disease, Tourette's syndrome and supranuclear palsy), neonatal cerebral hemorrhage, and spasticity.

Administration of effective amounts of a compound of formula I to a subject presenting with one or more of the foregoing symptom(s) will detectably decrease, eliminate, or prevent the subject symptom(s). In exemplary embodiments, administration of a compound of formula I to a suitable test subject will yield a reduction in one or more target symptom(s) associated with a neurological or psychiatric disorder by at least 10%, 20%, 30%, 50% or greater, up to a 75-90%, or 95% or greater, reduction in the one or more target symptom(s), compared to placebo-treated or other suitable control subjects. Comparable levels of efficacy are contemplated for the entire range of neurological and psychiatric disorders, and related conditions and symptoms, identified herein for treatment or prevention using the compositions and methods of the invention.

In exemplary embodiments of the invention, pharmaceutical compositions comprising a substituted 2-pyridinyl[7-(pyridin-4-yl)pyrazolo[1,5-a]pyrimidin-3-yl]methanones of formula I of the present invention are useful to prevent, reduce the severity of, or entirely reverse, mood disorders. As used herein, mood disorders include, but are not limited to, unipolar and bipolar depression, generalized anxiety disorder, and more specific anxiety disorders such as agoraphobia, panic disorder and social phobia, obsessive-compulsive disorder and post traumatic stress disorder. The compositions of the present invention are also useful in preventing and treating symptoms of anxiety disorders, including associated cardiac arrhythmias. The effectiveness of the compositions for these and related conditions can be routinely demonstrated according to a variety of methods, including, for example, by measuring markers such as those measured in the Clinician Administered PTSD Scale, the Eysenck Personality Inventory, the Hamilton Anxiety Scale, or in various animal models such as the well-known Vogel (thirsty rat conflict) test. Effective amounts of a compound of formula I will measurably prevent, decrease the severity of, or delay the onset or duration of, one or more of the foregoing mood disorders in a mammalian subject.

Within other exemplary embodiments, the pharmaceutical compositions containing the compounds of formula I of the present invention are particularly useful for the prevention of, reducing the development of, or reversal of, psychotic disorders. As used herein, psychotic disorders include, but are not limited to, mania; schizophrenia; paranoid, disorganized, catatonic, undifferentiated, or residual type schizophreniform disorder; schizoaffective disorder, including but not limited to delusional or depressive type; delusional disorder; brief psychotic disorder; shared psychotic disorder; psychotic disorder due to a general medical condition; substance-induced psychotic disorder, including but not limited to, psychosis induced by alcohol, amphetamine, cannabis, cocaine, hallucinogens, inhalants, opioids, or phencyclidine; personality disorder of the paranoid type; personality disorder of the schizoid type; and various other known and defined psychotic disorders, along with their associated neurological conditions and symptoms. Therapeutic and prophylactic efficacy of the compounds and methods of the invention for treating psychotic disorders can be demonstrated, for example, by measuring markers such as those determined within the Positive or Negative Syndrome Scale (PANSS), Scales for the Assessment of Negative Symptoms (SANS) or BPRS scores (Kay et al, Schizophrenia Bulletin 13:261-276, 1987), or in various animal models, such as in the “PCP or methamphetamine induced locomotor test” or the “conditioned avoidance response test.” Effective amounts of a compound of formula I will measurably prevent, decrease the severity of, or delay the onset or duration of, one or more of the foregoing psychotic disorders in a mammalian subject.

In other exemplary embodiments, the compositions and methods of the invention are effective to treat or prevent one or more symptom(s) of dementia of the Alzheimer's type, substance-induced delirium, and major depressive disorder with psychotic features.

In yet additional exemplary embodiments, the compositions and methods of the invention are effective to treat or prevent myotonia. Myotonia is a neuromuscular disorder characterized by the slow relaxation of the muscles. Symptoms may include muscle stiffness and hypertrophy (enlargement). The disorder is caused by a genetic mutation involving the chloride channel of the muscles. Effectiveness of the compositions of formula I in instances of myotonia may be determined, for example, by a measurable decrease or absence of stiffness and hypertrophy in treated subjects.

In further exemplary embodiments, the compounds of formula I are effective in preventing or ameliorating symptoms associated with stroke, cerebral ischemia, neonatal cerebral hemorrhage, and head trauma. It is predicted that GABA synthesis and release are decreased during an ischemic event. Accordingly, GABA agonists of the present invention will counteract the glutamatergic hyperactivity caused by cerebral ischemia, resulting in a neuroprotectant effect.

In another embodiment, the compounds of formula I are effective to treat or prevent pain, including neuropathic pain. It is widely thought that a lack of inhibition mediated by GABA is responsible for many pain states, including pain from nerve injury. Effective amounts of a compound of formula I will alleviate or prevent pain symptoms in mammalian subjects, including symptoms of neuropathic pain.

In yet another embodiment, compositions and methods of the invention are useful to treat movement disorders. A movement disorder is a neurological disturbance that involves one or more muscles or muscle groups. Movement disorders include Parkinson's disease, Huntington's Chorea, progressive supranuclear palsy, myoclonus, spasticitiy, Wilson's disease, Tourette's syndrome, epilepsy, and various chronic tremors, seizures, tics and dystonias. Tremors are characterized by abnormal, involuntary movements. An essential tremor is maximal when the body part afflicted (often an arm or hand) is being used, for example when attempts at writing or fine coordinated hand movements are made. Dystonias are involuntary movement disorders characterized by continued muscular contractions which can result in twisted contorted postures involving the body or limbs. Particular dystonias can include spasmodic torticollis, blepharospasm and writer's cramp. Tic disorders (including Tourette's syndrome) are usually very rapid, short-lived, stereotyped repeated movements. The more common tics involve the motor systems, or are vocal in nature. Motor tics often involve the eyelids, eyebrows or other facial muscles, as well as the upper limbs. Vocal tics may involve grunting, throat clearing, coughing or cursing. Individuals with tic disorders will often describe a strong urge to perform the particular tic, and may actually feel a strong sense of pressure building up inside of them if the action is not performed. Effective amounts of a compound of formula I will decrease involuntary movements and seizures in mammalian subjects. Applying the above-noted therapeutic index expressing treatment efficacy for all therapeutic compositions and methods of the invention, test subjects will exhibit a 10%, 20%, 30%, 50% or greater reduction, up to a 75-90%, or 95% or greater, reduction in one or more symptoms associated with a movement disorder compared to placebo-treated or other suitable control subjects.

Anticonvulsant efficacy of compounds and methods of the invention can be demonstrated using various accepted animal models predictive of activity in humans, including, but not limited to, the maximal electroshock (MES) model (human analog: tonic-clonic seizures) as described in Krall et al. (1978), the threshold pentylenetetrazole (PTZ) model (human analog: absence seizures) as described in Krall et al. (1978), the amygdala-kindling model (human analog: complex partial seizures with secondary generalization) as described in Albright & Burnham (1980), and the AY-9944 model (human analog, a typical absence, a component of the Lennox-Gastaut syndrome) as described in Cortez et al. (2001). Therapeutic efficacy and acceptable toxicity will also be demonstrable using standard assays, such as the standard rotorod assay as described in Dunham & Miya (1957) and Wlaz & Loscher (1998). Based on the results of anticonvulsant assays (typically expressed in units of ED₅₀) and toxicity assays (typically expressed in units of TD₅₀), a therapeutic index will be calculable for each compound of the invention, e.g., as a ratio of TD₅₀/ED₅₀. The larger the therapeutic index, the more desirable the compound for use in the methods of the invention.

Within exemplary embodiments of the invention, GABA and GABA_{A} receptor modulating compositions are provided, including pharmaceutical compositions that mediate their effects by modulating a function or activity of GABA, or of a GABA_{A} receptor. These compositions are effective for prophylaxis or treatment of neurological and psychological disorders mediated by a deficiency, defect, or imbalance in GABA or GABA_{A} receptor physiology in mammalian subjects. Within exemplary embodiments, the compositions of the invention are effective within in vivo treatment methods to alleviate or prevent one or more symptoms of a psychiatric or neurological disorder selected from stroke, head trauma, epilepsy, pain, migraine, mood disorders, anxiety, post traumatic stress disorder, bi-polar disorder, obsessive compulsive disorders, schizophrenia, seizures, convulsions, tinnitus, neurodegenerative disorders including Alzheimer's disease, Huntington's chorea, amyotrophic lateral sclerosis and Parkinson's disease, depression, bipolar disorders, mania, trigeminal and other neuralgia, neuropathic pain, hypertension, cerebral ischemia, cardiac arrhythmia, myotonia, cocaine abuse, myoclonus, essential tremor, dyskinesia and other movement disorders, neonatal cerebral hemorrhage, and spasticity, among other neurological and psychological diseases, conditions, and disorders.

GABA and GABA_{A} receptor modulating compositions of the invention typically comprise a GABA or GABA_{A} receptor modulating effective amount of a 2- and 4-pyridinyl substituted 2-pyridinyl[7-(pyridin-4-yl)pyrazolo[1,5-a]pyrimidin-3-yl]methanone of formula I. The active compound may be optionally formulated with a pharmaceutically acceptable carrier and/or various excipients, vehicles, stabilizers, buffers, preservatives, etc. An “effective amount,” “therapeutic amount,” “therapeutic effective amount,” or “effective dose” is an effective amount or dose sufficient to elicit a desired pharmacological or therapeutic effect in a mammalian subject—typically resulting in a measurable reduction in an occurrence, frequency, or severity of one or more symptom(s) associated with or caused by a neurological or psychological disease, condition, or disorder in the subject. In certain embodiments, when a compound of the invention is administered to treat a central nervous system (CNS) disorder, an effective amount of the compound will be an amount sufficient to pass across the blood-brain barrier of the subject and interact functionally with GABA or GABA_{A} receptors at CNS sites. In more detailed embodiments, prevention of a neurological or psychiatric condition or disorder will manifest by delaying or eliminating onset of symptoms of the condition or disorder. Therapeutic efficacy can alternatively be demonstrated by decrease in the frequency or severity of symptoms associated with the treated condition or disorder, or by altering the nature, recurrence, or duration of symptoms associated with the treated condition or disorder. Therapeutically effective amounts, and dosage regimens, of the compositions of formula I, including pharmaceutically effective salts, solvates, hydrates, polymorphs or prodrugs thereof, will be readily determinable by those of ordinary skill in the art, often based on routine clinical or patient-specific factors.

Suitable routes of administration for GABA_{A} receptor modulating compositions of the invention comprising 2- and 4-pyridinyl substituted 2-pyridinyl[7-(pyridin-4-yl)pyrazolo[1,5-a]pyrimidin-3-yl]methanones include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Suitable effective unit dosage amounts of a 2- and 4-pyridinyl substituted 2-pyridinyl[7-(pyridin-4-yl)pyrazolo[1,5-a]pyrimidin-3-yl]methanone of formula I for mammalian subjects may range from about 1 to 1800 mg, 50 to 1000 mg, 75 to 900 mg, 100 to 750 mg, or 150 to 500 mg. In certain embodiments, the effective dosage will be selected within narrower ranges of, for example, 10 to 25 mg, 30-50 mg, 75 to 100 mg, 100 to 250 mg, or 250 to 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In exemplary embodiments, dosages of 10 to 25 mg, 30-50 mg, 75 to 100 mg, 100 to 250 mg, or 250 to 500 mg, are administered one, two, three, or four times per day. In more detailed embodiments, dosages of 50-75 mg, 100-200 mg, 250-400 mg, or 400-600 mg are administered once or twice daily. In alternate embodiments, dosages are calculated based on body weight, and may be administered, for example, in amounts from about 0.5 mg/kg to about 20 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day.

The amount, timing and mode of delivery of compositions of the invention comprising an effective amount of a 2- and 4-pyridinyl substituted 2-pyridinyl[7-(pyridin-4-yl)pyrazolo[1,5-a]pyrimidin-3-yl]methanone of formula I will be routinely adjusted on an individual basis, depending on such factors as weight, age, gender, and condition of the individual, the acuteness of the incontinence and/or related symptoms, whether the administration is prophylactic or therapeutic, and on the basis of other factors known to effect drug delivery, absorption, pharmacokinetics, including half-life, and efficacy. An effective dose or multi-dose treatment regimen for the compounds of the invention will ordinarily be selected to approximate a minimal dosing regimen that is necessary and sufficient to substantially prevent or alleviate one or more symptom(s) of a neurological or psychiatric condition in the subject, as described herein. Thus, following administration of the a 2- and 4-pyridinyl substituted 2-pyridinyl[7-(pyridin-4-yl)pyrazolo[1,5-a]pyrimidin-3-yl]methanone according to the formulations and methods of the invention, test subjects will exhibit a 10%, 20%, 30%, 50% or greater reduction, up to a 75-90%, or 95% or greater, reduction, in one or more symptoms associated with a targeted psychiatric or neurological disorder, compared to placebo-treated or other suitable control subjects.

Within additional aspects of the invention, combinatorial formulations and coordinate administration methods are provided which employ an effective amount of one or more compounds of formula I, and one or more additional active agent(s) that is/are combinatorially formulated or coordinately administered with the compound of formula I-yielding an effective formulation or method to modulate a function or activity of GABA, or of a GABA_{A} receptor, and/or to alleviate or prevent one or more symptom(s) of a neurological or psychiatric disorder in a mammalian subject. Exemplary combinatorial formulations and coordinate treatment methods in this context employ a compound of formula I in combination with one or more additional or adjunctive anxiolytic, antidepressant, anticonvulsant, nootropic, anesthetic, hypnotic or muscle relaxant agent(s). In additional combinatorial formulations and coordinate treatment methods, a 2- and 4-pyridinyl substituted 2-pyridinyl[7-(pyridin-4-yl)pyrazolo[1,5-a]pyrimidin-3-yl]methanone compound is formulated or co-administered in combination with one or more secondary therapeutic agents used to treat symptoms which may accompany the psychiatric or neurological conditions listed above.

To practice the coordinate administration methods of the invention, a 2- and 4-pyridinyl substituted 2-pyridinyl[7-(pyridin-4-yl)pyrazolo[1,5-a]pyrimidin-3-yl]methanone compound is administered, simultaneously or sequentially, in a coordinate treatment protocol with one or more of the secondary or adjunctive therapeutic agents contemplated herein. The coordinate administration may be done simultaneously, or sequentially in either order, and there may be a time period while only one or both (or all) active therapeutic agents, individually and/or collectively, exert their biological activities. A distinguishing aspect of all such coordinate treatment methods is that the 2- and 4-pyridinyl substituted 2-pyridinyl[7-(pyridin-4-yl)pyrazolo[1,5-a]pyridin-3-yl]methanone compound exerts at least some detectable GABA or GABA_{A} receptor modulating activity, and/or elicits a favorable clinical response, which may or may not be in conjunction with a secondary clinical response provided by the secondary therapeutic agent. Often, the coordinate administration of a 2- and 4-pyridinyl substituted 2-pyridinyl[7-(pyridin-4-yl)pyrazolo[1,5-a]pyrimidin-3-yl]methanone compound with a secondary therapeutic agent as contemplated herein will yield an enhanced therapeutic response beyond the therapeutic response elicited by either or both the 2- and 4-pyridinyl substituted 2-pyridinyl[7-(pyridin-4-yl)pyrazolo[1,5-a]pyrimidin-3-yl]methanone compound and/or secondary therapeutic agent alone.

Pharmaceutical dosage forms of the 2- and 4-substituted 2-pyridinyl[7-(pyridin4-yl)pyrazolo [1,5-a]pyrimidin-3-yl]methanones of the present invention include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

The compositions of the invention for treating neurological and psychiatric disorders can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents will often be biologically inactive and can be administered to patients without causing deleterious side effects or interactions with the active agent.

If desired, the 2- and 4-substituted 2-pyridinyl[7-(pyridin-4-yl)pyrazolo[1,5-a]pyrimidin-3-yl]methanones of the invention can be administered in a controlled release form by use of a slow release carrier, such as a hydrophilic, slow release polymer. Exemplary controlled release agents in this context include, but are not limited to, hydroxypropyl methyl cellulose, having a viscosity in the range of about 100 cps to about 100,000 cps.

2- and 4-pyridinyl substituted 2-pyridinyl[7-(pyridin-4-yl)pyrazolo[1,5-a]pyrimidin-3-yl]methanone compositions of the invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, which may be prepared by any conventional method of preparing pharmaceutical oral unit dosage forms can be utilized in preparing oral unit dosage forms. Oral unit dosage forms, such as tablets, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate. Such rapidly acting dosage forms would be useful, for example, in the prevention or treatment of acute attacks of panic disorder.

Additional 2- and 4-pyridinyl substituted 2-pyridinyl[7-(pyridin-4-yl)pyrazolo[1,5-a]pyrimidin-3-yl]methanone compositions of the invention can be prepared and administered in any of a variety of inhalation or nasal delivery forms known in the art. Devices capable of depositing aerosolized 2- and 4-pyridinyl substituted 2-pyridinyl[7-(pyridin-4-yl)pyrazolo[1,5-a]pyrimidin-3-yl]methanones formulations in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of seizures or panic disorder. Methods and compositions suitable for pulmonary delivery of drugs for systemic effect are well known in the art. Suitable formulations wherein the carrier is a liquid for administration, as for example, a nasal spray or as nasal drops, may include aqueous or oily solutions of 2- and 4-pyridinyl substituted 2-pyridinyl[7-(pyridin-4-yl)pyrazolo[1,5-a]pyrimidin-3-yl]methanones and any additional active or inactive ingredient(s).

Intranasal delivery permits the passage of such a compound to the blood stream directly after administering an effective amount of the compound to the nose, without requiring the product to be deposited in the lung. In addition, intranasal delivery can achieve direct, or enhanced, delivery of the active compound to the CNS. In these and other embodiments, intranasal administration of the compounds of the invention may be advantageous for treating sudden onset anxiety disorders, such as panic disorder. Typically, the individual suffering from generalized anxiety disorder and prone to attacks of panic disorder is able to sense when such an attack is imminent. At such times, being able to administer compounds of the invention in a form that is convenient, even in a public setting, and yields rapidly absorption and CNS delivery, is particularly desirable.

For intranasal and pulmonary administration, a liquid aerosol formulation will often contain an active compound of the invention combined with a dispersing agent and/or a physiologically acceptable diluent. Alternative, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle suitable of a sufficiently small particle diameter, e.g., in a range of from about 2-5 microns, for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional compositions and methods of the invention are provided for topical administration of 2- and 4-pyridinyl substituted 2-pyridinyl[7-(pyridin-4-yl)pyrazolo[1,5-a]pyrimidin-3-yl]methanones for treating neurological and psychiatric disorders associated with GABA and GABA_{A} receptors. Topical compositions may comprise 2- and 4-pyridinyl substituted 2-pyridinyl[7-(pyridin-4-yl)pyrazolo[1,5-a]pyrimidin-3-yl]methanones and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise 2- and 4-pyridinyl substituted 2-pyridinyl[7-(pyridin-4-yl)pyrazolo[1,5-a]pyrimidin-3-yl]methanones dissolved or dispersed in a portion of a water or other solvent or liquid to be incorporated in the topical composition or delivery device. It can be readily appreciated that the transdermal route of administration may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example 24 hours. A once-daily transdermal patch is particularly useful for a patient suffering from generalized anxiety disorder.

Yet additional 2- and 4-pyridinyl substituted 2-pyridinyl[7-(pyridin-4-yl)pyrazolo[1,5-a]pyrimidin-3-yl]methanone formulations are provided for parenteral administration, including aqueous and non-aqueous sterile injection solutions which may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers. 2- and 4-pyridinyl substituted 2-pyridinyl[7-(pyridin-4-yl)pyrazolo[1,5-a]pyrimidin-3-yl]methanone formulations may also include polymers for extended release following parenteral administration. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

In more detailed embodiments, 2- and 4-pyridinyl substituted 2-pyridinyl[7-(pyridin-4-yl)pyrazolo[1,5-a]pyrimidin-3-yl]methanones may be encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macro emulsions.

The pharmaceutical agents of the invention may be administered parenterally, e.g. intravenously, intramuscularly, subcutaneously or intraperitoneally. The parenteral preparations may be solutions, dispersions or emulsions suitable for such administration. The subject agents may also be formulated into polymers for extended release following parenteral administration. Pharmaceutically acceptable formulations and ingredients will typically be sterile or readily sterilizable, biologically inert, and easily administered. Such polymeric materials are well known to those of ordinary skill in the pharmaceutical compounding arts. Parenteral preparations typically contain buffering agents and preservatives, and may be lyophilized to be re-constituted at the time of administration.

The following examples illustrate certain embodiments of the present invention, and are not to be construed as limiting the present disclosure. The evidence provided in these examples demonstrates that 2-pyridinyl[7-(pyridin-4-yl)pyrazolo[1,5-a]pyrimidin-3-yl]methanone compounds of the invention, substituted on the 2- and 4-pyridinyl moieties and represented by formiula I, are effective modulators of GABAA receptor physiology useful for treating neurological and psychiatric disorders in mammals.

### EXAMPLE 1

### Synthesis of (5-amino-1H-pyrazol-4-yl)-(6-methylpyridin-2-yl)-methanone

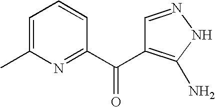

Sodium (0.62 g, 0.0269 moles) and anhydrous ethanol (30 ml) were combined and stirred until complete dissolution. The solvent was removed under reduced pressure followed by azeotroping with anhydrous toluene (11 ml) to yield a white powder. Toluene (11 ml) was then added to the white powder followed by ethyl-6-methylpyridinecarboxylate (5 g, 0.0269 moles) and anhydrous acetonitrile (1.84 ml, 0.039 moles). The reaction was stirred for 30 minutes at reflux at which time the mixture became so thick it would not stir. Additional toluene was added (11 ml) and reflux was continued for 2 hours. The reaction was cooled to room temperature and heptane (120 ml) added. The solid was filtered and dried to isolate the crude product. Liquid chromatography/mass spectrometry (LC/MS) M ⁺H 161. The solid was then suspended in dichloromethane (50 ml) and acidified with acetic acid. The mixture was filtered through a silica gel plug eluted with dichloromethane followed by 10% acetonitrile/dichloromethane. The fractions containing the product were concentrated to an orange oil which crystallized upon setting to give 3-(6-methylpyridin-2-yl)-3-oxopropionitrile (3.14 g, 92% yield) which was used without further purification.

The 3-(6-methylpyridin-2-yl)-3-oxopropionitrile (3.14 g, 0.0196 moles) and dichloromethane (30 ml) were then combined and cooled to −10° C. Next, dimethylformamide dimethylacetal (2.57 g, 0.0216 moles) was added. Thin layer chromatography (TLC) showed the reaction was complete after 2 hours. The reaction was concentrated to a brown oil. LC/MS M+H 216. The brown oil was then dissolved in ethanol (25 ml) with aminoguanidine nitrate (3.41 g, 0.0249 moles) and 10 N sodium hydroxide (1.7 ml). After 4 hours at reflux, LC/MS showed that the reaction was complete. The solvent was then removed under reduced pressure and the residue dissolved in ethyl acetate and washed with water (2×), dried over magnesium sulfate, filtered and concentrated to a brown solid which was identified as (3-amino-1H-pyrazol-4-yl)-(6-methylpyridin-2-yl)-methanone (2.65 g, 67% yield). LC/MS M+H 203

### EXAMPLE 2

### Synthesis of (5-amino-1H-pyrazol-4-yl)-(5-methylpyridin-2-yl)-methanone

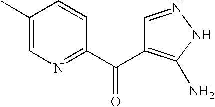

Sodium (2.2 g, 0.0963 moles) and anhydrous ethanol were combined and stirred until dissolved. The ethanol and azeotrope were stripped off with anhydrous toluene (35 ml). Additional anhydrous toluene (35 ml) was added followed by ethyl-5-methylpyridinecarboxylate (15.9 g, 0.0963 moles) and anhydrous acetonitrile (6.6 ml, 0.125 moles). The mixture was refluxed for 5 hrs then stirred at room temperature overnight. The reaction was then diluted with heptane (300 ml) and the solid was filtered and dried. LC/MS M ⁺H 161. The solid was then suspended in dichloromethane (150 ml) and acidified with acetic acid. The mixture was filtered through a silica gel plug with dichloromethane. The organic portions were removed to yield a dark brown solid, 3-(5-methylpyridin-2-yl)-3-oxopropionitrile (12.98 g, 84% yield), which was used without further purification.

Next, 3-(5-methylpyridin-2-yl)-3-oxopropionitrile (12.98 g, 0.081 moles) and dichloromethane (150 ml) were combined and cooled to −10° C. Dimethylformamide dimethylacetal (2.57 g, 0.0216 moles) was added. TLC showed the reaction was complete after 2 hours. The reaction was concentrated to a brown oil. LC/MS M ⁺H 216. Next, the brown oil was dissolved in ethanol (125 ml) with aminoguanidine nitrate (14.1 g, 0.103 moles) and 10 N sodium hydroxide (8.5 ml). After 5 hours at reflux the reaction was cooled to room temperature and stirred overnight. LC/MS showed that the reaction was complete. The solvent was stripped off and the residue dissolved in ethyl acetate and washed with water (2×), dried over magnesium sulfate, filtered and stripped to a brown solid which was identified as (3-amino-1H-pyrazol-4-yl)-(5-methylpyridin-2-yl)-methanone (5.27 g, 32% yield). LC/MS M ⁺H 203. ¹H NMR (DMSO) 2.39 (3H, s, CH₃), 6.88 (2H, br, s), 7.8 (1H, d), 7.9 (1H, d), 8.32 (1H, s), 856 (1H, s), 11.88 (1H, br, s).

### EXAMPLE 3

### General Procedure for Weinreb Amide Synthesis

1,1′-carbonyldiimidazole (1.1 eq) was added to a stirring solution of carboxylic acid (1 eq) in dichloromethane and the mixture was stirred for 2 hours at room temperature. N,O-dimethylhydroxylamine hydrochloride (1.5 equ) was then added and the mixture was stirred overnight. The mixture was quenched, and then partitioned with sodium hydroxide (0.1N). The organic layer was washed with brine, dried over magnesium sulfate, filtered and concentrated under reduced pressure. Generally, the Weinreb amide was used as is, if necessary silica gel chromatography was conducted.

### EXAMPLE 4

### General Procedure for Synthesis of Acetyl Pyridine from Weinreb Amide

The Weinreb amide (1 eq), as produced in Example 3, was dissolved in anhydrous tetrahydrofuran and cooled to 0-5° C. Methylmagnesium iodide (3.0 M, 1.3 eq) was added dropwise. After 2 hours, the reaction was quenched with saturated ammonium chloride and extracted with ethyl acetate. The organic portion was dried over magnesium sulfate, filtered and concentrated under reduced pressure. The crude material was purified, if needed, by silica gel chromatography eluted with heptane and ethyl acetate as the eluent.

### EXAMPLE 5

### General Procedure for Synthesis of enamine

The acetyl pyridine (1.0 eq) and dimethylformamide dimethylacetal (6.0 eq) were heated to reflux and stirred for 5 hours. The reaction was then concentrated, filtered through packed florisil (2 g SPE) eluting with methylene chloride (200 ml). The methylene chloride solution was then concentrated to yield the target enamine compound. The products were characterized by the olefinic protons using NMR.

### EXAMPLE 6

### Synthesis of [7-(2-chloro-pyridin-4-yl)-pyrazolo[1,5-a]pyrimidin-3-yl]-(5-methyl-pyridin-2-yl)-methanone

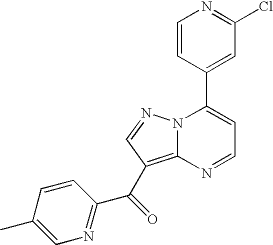

1-(2-chloro-pyridin-4-yl)-3-dimethylamino-propenone (1.18 g, 0.0056 moles) and (3-amino-1H-pyrazol-4-yl)-(5-methyl-pyridin-2-yl)-methanone (0.56 g, 0.0028 moles) were added to acetic acid (5 ml). The mixture was heated to reflux until the reaction was judged complete by TLC (6 hours). The reaction mixture was then poured into water and the resulting solid filtered, washed with water and dried. Purification was done using a 40s Biotage column eluting with chloroform:methanol (99:1). Further purification was done by triturating with methylene chloride and heptanes to yield [7-(2-Chloro-pyridin-4-yl)-pyrazolo[1,5-a]pyrimidin-3-yl]-(5-methyl-pyridin-2-yl)-methanone, an off-white solid (493 mg, 50%). 97% pure by high performance liquid chromatography (HPLC) area percent. M/e+350. ¹H NMR (CDCl₃) 2.48 (3H, s, CH₃), 7.20 (1H, d), 7.75 (1H, d), 7.92 (1H, dd), 8.04 (1H, dd), 8.19 (1H, d), 8.59 (1H, s), 8.69 (1H, d), 8.95 (1H, d), 9.42 (1H, s).

### EXAMPLE 7

### Synthesis of [7-(2-bromo-pyridin-4-yl)-pyrazolo[1,5-a]pyrimidin-3-yl]-(5-methyl-pyridin-2-yl)-methanone

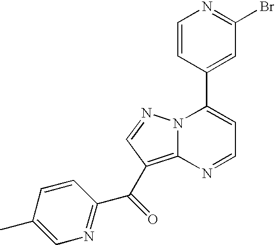

1-(2-bromo-pyridin-4-yl)-3-dimethylamino-propenone (1.14 g, 0.0045 moles) and (3-amino-1H-pyrazol-4-yl)-(5-methyl-pyridin-2-yl)-methanone (0.45 g, 0.0022 moles) were added to acetic acid (5 ml). The mixture was heated to reflux until the reaction was judged complete by TLC (6 hours). The reaction mixture was poured into water and the resulting solid filtered, washed with water and dried. Purification was done using a 40s Biotage column eluting with chloroform:methanol (99:1). Further purification was done by triturating with methylene chloride and heptanes to yield [7-(2-bromo-pyridin-4-yl)-pyrazolo[1,5-a]pyrimidin-3-yl]-(5-methyl-pyridin-2-yl)-methanone, a light yellow solid (486 mg, 56%). 95% pure by HPLC area percent. M/e+395. ¹H NMR (CDCl₃) 2.43 (3H, s, CH₃), 7.20 (1H, d), 7.75 (1H, d), 7.96 (1H, dd), 8.19 (2H, dd), 8.59 (1H, s), 8.66 (1H, d), 8.95 (1H, d), 9.42 (1H, s).

### EXAMPLE 8

### Synthesis of [7-(2-benzoyl-pyridin-4-yl)-pyrazolo[1,5-a]pyrimidin-3-yl]-(5-methyl-pyridin-2-yl)-methanone

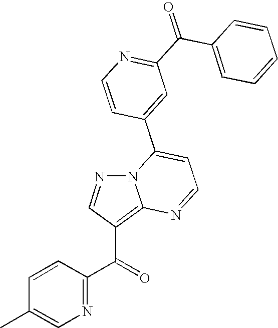

1-(2-benzoyl-pyridin-4-yl)-3-dimethylamino-propenone (113 g, 0.0040 moles) and (3-amino-1H-pyrazol-4-yl)-(5-methyl-pyridin-2-yl)-methanone (0.41 g, 0.0020 moles) were added to acetic acid (5 ml). The mixture was heated to reflux until the reaction was judged complete by TLC (5 hours). The reaction mixture was poured into water and the resulting solid filtered, washed with water and dried. Purification was done using a 40s Biotage column eluting with chloroform:methanol (99:1). Further purification was done by triturating with methylene chloride and heptanes to yield [7-(2-Benzoyl-pyridin-4-yl)-pyrazolo[1,5-a]pyrimidin-3-yl]-(5-methyl-pyridin-2-yl)-methanone, an off-white solid (362 mg, 44%). 98% pure by HPLC area percent. M/e+420. ¹H NMR (CDCl₃) 2.53 (3H, s, CH₃), 7.30 (1H, d), 7.54 (2H, dd), 7.66 (1H, dd), 7.77 (1H, d), 8.22 (3H, m), 8.29 (1H, d), 8.64 (2H, m), 9.02 (2H, dd), 9.42 (1H, s).

### EXAMPLE 9

### Synthesis of [7-(2-chloro-pyridin-4-yl)-pyrazol of [1,5-a]pyrimidin-3-yl]-(6-methyl-pyridin-2-yl)-methanone

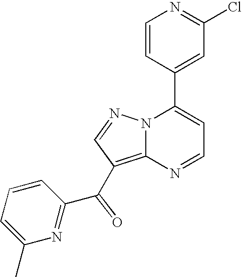

1-(2-chloro-pyridin-4-yl)-3-dimethylamino-propenone (1.28 g, 0.0061 moles) and (3-amino-1H-pyrazol-4-yl)-(6-methyl-pyridin-2-yl)-methanone (0.61 g, 0.0030 moles) were added to acetic acid (5 ml). The mixture was heated to reflux until the reaction was judged complete by TLC (5 hours). The reaction mixture was poured into water and the resulting solid filtered, washed with water and dried. Purification was done using a 40s Biotage column eluting with chloroform:methanol (99:1). Further purification was done by triturating with methylene chloride and heptanes to yield [7-(2-chloro-pyridin-4-yl)-pyrazolo[1,5-a]pyrimidin-3-yl]-(6-methyl-pyridin-2-yl)-methanone, an off-white solid (250 mg, 24%). 95% pure by HPLC area percent. M/e+350. ¹H NMR (CDCl₃) 2.75 (3H, s, CH₃), 7.21 (1H, d), 7.41 (1H, d), 7.82 (1H, dd), 7.92 (1H, d), 8.05 (1H, s), 8.09 (1H, d), 8.69 (1H, d), 8.95 (1H, d), 9.50 (1H, s).

### EXAMPLE 10

### Synthesis of [7-(2-bromo-pyridin-4-yl)-pyrazolo[1,5-a]pyrimidin-3-yl]-(6-methyl-pyridin-2-yl)-methanone

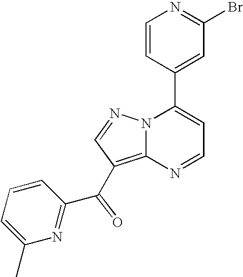

1-(2-bromo-pyridin-4-yl)-3-dimethylamino-propenone (1.10 g, 0.0043 moles) and (3-amino-1H-pyrazol-4-yl)-(6-methyl-pyridin-2-yl)-methanone (0.43 g, 0.0022 moles) were added to acetic acid (5 ml). The mixture was heated to reflux until the reaction was judged complete by TLC (6 hours). The reaction mixture was poured into water and the resulting solid filtered, washed with water and dried. Purification was done using a 40s Biotage column eluting with chloroform:methanol (99:1). Further purification was done by triturating with methylene chloride and heptanes to yield [7-(2-bromo-pyridin-4-yl)-pyrazolo[1,5-a]pyrimidin-3-yl]-(6-methyl-pyridin-2-yl)-methanone, a light yellow solid (240 mg, 30%). 99% pure by HPLC area percent. M/e+395. ¹H NMR (CDCl₃) 2.68 (3H, s, CH₃), 7.19 (1H, d), 7.38 (1H, d), 7.81 (1H, dd), 7.94 (1H, d), 8.07 (1H, d), 8.17 (1H, s), 8.65 (1H, d), 8.95 (1H, d), 9.5 (1H, s).

### EXAMPLE 11

### Synthesis of [7-(2-benzoyl-pyridin-4-yl)-pyrazolo[1,5-a]pyrimidin-3-yl]-(6-methyl-pyridin-2-yl)-methanone

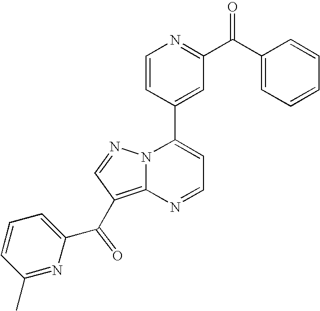

1-(2-benzoyl-pyridin-4-yl)-3-dimethylamino-propenone (1.14 g, 0.0041 moles) and (3-amino-1H-pyrazol-4-yl)-(6-methyl-pyridin-2-yl)-methanone (0.40 g, 0.0020 moles) were added to acetic acid (5 ml). The mixture was heated to reflux until the reaction was judged complete by TLC (5 hours). The reaction mixture was poured into water and the resulting solid filtered, washed with water and dried. Purification was done using a 40s Biotage column eluting with chloroform:methanol (99:1). Further purification was done by triturating with methylene chloride and heptanes to yield [7-(2-benzoyl-pyridin-4-yl)-pyrazolo[1,5-a]pyrimidin-3-yl]-(6-methyl-pyridin-2-yl)-methanone, an off-white solid (241 mg, 29%). 96% pure by HPLC area percent. M/e+420. ¹H NMR (CDCl₃) 2.71 (3H, s, CH₃), 7.32 (1H, d), 7.45 (1H, d), 7.55 (2H, t), 7.69 (1H, t), 7.84 (1H, t), 8.10 (1H, d), 8.17 (2H, m), 8.29 (1H, d), 8.66 (1H, s), 9.01 (2H, m), 9.52 (1H, s).

### EXAMPLE 12

### Binding Assay for Compounds of Formula I

The assay conditions employed for the present example are modified as indicated from Skolnick, et al., J. Pharmacol. Exper. Ther. 283:488-493, 1997, and Liu, et al., J. Med. Chem. 39:1928-1934, 1996. Tissue Preparation: Cerebella obtained from adult male Sprague Dawley rats (or equivalent strain) were killed by decapitation. The brains were immediately removed and placed in beakers containing ice-cold 50 mM Tris-citrate buffer, pH 7.4. After weighing, the tissues were disrupted in 50 volumes of ice cold Tris-Citrate buffer using a Polytron (setting 6-7, 20 sec) (or equivalent tissue disruptor). The homogenates were centrifuged at 20,000×g (4° C.) for 20 min. The supernatants were discarded and the resulting pellets resuspended in an equal volume of buffer and recentrifuged. This washing procedure was repeated a total of five times. Following the final resuspension, the tissue was resuspended in the Tris-citrate buffer and frozen on solid carbon dioxide. The frozen tissue was maintained at −70° C. for three to five days before the assay was conducted. The purpose of this extensive washing procedure is to eliminate endogenous GABA from the tissue preparation. Thus, the effects of adding GABA back to the assay on ligand affinity can be determined (the so called GABA-shift assay). Incubation volumes were generally selected at 1 ml. Tissue suspension: 0.1 ml (50-100 ug protein), NaCl (2M): 0.1 ml (to yield 200 mM final) (stock soln. in assay buffer), [3H]Ro 15-1788: 0.05 ml (to yield final concentration of ˜1 nM), Drugs or buffer: 0.05 ml, GABA (final, 10 μM): 0.05 ml<only in those assays requiring a Buffer: to 1 ml (i.e., 0.7 ml). Incubations were performed for 120 min. on ice, and terminated by rapid filtration through Whatman GF/B filters with 2×5 ml washes of ice-cold Tris-Citrate buffer. The radioactivity retained by the filters was counted by standard liquid scintillation spectroscopy. Nonspecific binding can be defined with diazepam (final, 5-10 uM), flunitrazepam (5-10 uM), or Ro 15-1788 (1-5 uM) (these can be made up in a stock solution in ethanol and diluted in buffer to a 20×. For diazepam, e.g., make up 10 mM, dilute this in buffer to 100 uM, and use 50 ul of 100 uM in 1 ml final incubation volume). Nonspecific binding is usually no more than 10-15% of total binding under these conditions. After harvesting, the cerebella were weighed, homogenized in 50 volumes of ice-cold 50 mM Tris-citrate buffer (pH 7.4) and centrifuged at 20,000×g for 20 minutes. The homogenization and centrifugation steps (“washing”) were repeated five times. The results are shown in Table 1 above.

The results of these assays, provided in Table 1, indicate that several of the exemplary compounds of the invention exhibit good affinity for the GABA_{A} receptor, as demonstrated by their ability to inhibit [³H]Ro 15-1788 binding to the receptor preparation with an IC₅₀ of less than 10 μM.

Alternative competition binding assays can be performed using the selective radioligand [³H]Ro 15-1788. The wells of an MAFBNOB multiwell plate (Millipore Corp) are filled with an aliquot of a cerebellar membrane preparation (containing ˜0.1 mg total protein), an aliquot of [³H]Ro 15-1788 (1 nM, New England Nuclear), varying concentrations of the compound to be tested, and sufficient buffer to yield a total volume of 0.3 ml in each well. Nonspecific binding is assessed in separate wells by the addition of unlabeled diazepam (10 μM). The assay is incubated for 2 hours at 0-4° C., after which the incubation is terminated by vacuum filtration (Millipore Corp). The filters used in terminating the assay and collecting the radiolabeled receptors are washed twice with 0.3 ml of ice-cold buffer, placed into scintillation vials with 4.0 ml of liquid scintillation cocktail and the radioactivity present on the filters measured in a Beckman LS-6500 Scintillation Counter.

### EXAMPLE 13

### Demonstration of Anti-Anxiety and Anticonvulsant Properties

The anti-anxiety and anticonvulsant properties of the novel compounds of the present invention are established by conventional assays which assess anxiolytic and/or antiepileptic activity, for example by measuring protection from convulsions resulting from the administration of pentylenetetrazole. Single or graded dose levels of the test compounds are administered orally or intraperitoneally in a 2% starch vehicle, containing 0.5% v/v polyethylene glycol and one drop of Polysorbate 80 to groups of at least 4 rats. At 30 or 60 minutes, the rats are treated intravenously with pentylenetetrazole at a dose of 23 mg/kg of body weight. This dose is estimated to cause clonic seizures in 99% of unprotected rats. It has been reported (R. T. Hill and D. H. Tedeschi, “Animal Testing and Screening Procedures in Evaluating Psychotropic Drugs” in “An Introduction to Psychopharmacology”, Eds. R. R. Rech and K. E. Moore, Raven Press, New York, pp 237-288 (1971)) that there is a high degree of correlation between antagonism of pentylenetetrazole seizures in rats and anti-anxiety or anticonvulsant effects in higher warm-blooded animals such as mammals.

### EXAMPLE 14

### Demonstration of Anti-Anxiety Effects

Groups of 6 naive, Wistar strain rats, weighing 200-240 g each are deprived of water for 48 hours and food for 24 hours. The compounds of the invention are administered in single or graded, oral or intraperitoneal doses, suspended in a 2% starch vehicle containing 0.5% v/v polyethylene glycol and one drop of polysorbate 80. Control animals receive the vehicle alone. At 30 to 60 minutes each rat is placed in an individual plexiglass chamber. Water is available ad libitum from a tap located in the rear of the chamber. A 0.7 milliampere DC shocking current is established between the stainless steel grid floor and the tap. After 20 licks of non-shocked drinking, a shock is delivered for 2 seconds and then further shocks are delivered on a ratio of one shock for 2 seconds for every 20 licks. This is continued for a total of 3 minutes. The number of shocks taken by each rat during the 3 minute interval is recorded and compared to a control group. The compounds are considered active if the number of shocks received by the test group is significantly higher than the control group by the Mann-Witney U test.

### EXAMPLE 15

### Inhibition Binding of ³H-Benzodiazepine to Brain-Specific Receptors of Rats

Whole cortex of rats are homogenized gently in 20 volumes of ice-cold 0.32M sucrose, centrifuged twice at 1000 g for 10 minutes and then recentrifuged at 30,000 g for 20 minutes to produce a crude P₂-synaptosomal fraction. The P₂-fraction is either: (1) resuspended in twice the original volume in hypotonic 50 mM Tris.HCl (pH 7.4), or (2) resuspended in one-half the original volume in hypotonic 10 mM Tris.HCl (pH 7.4) and frozen (−20° C.) until time of use. Frozen P₂ preparations are thawed and resuspended in four times the original homogenizing volume at time of assay.

The binding assay consists of 300 μl of the P₂-fraction suspension (0.2-0.4 mg protein), 100 μl of test drug and 100 μl of ³H-diazepam (1.5 nM, final concentration) or ³H-flunitrazepam (1.0 nM, final concentration) which is added to 1.5 ml of 50 mM Tris.HCl (pH 7.4). Nonspecific binding controls and total binding controls receive 100 μl of diazepam (3 μM, final concentration) and 100 μl of deionized water, respectively, in place of the test compound. Incubation for 30 minutes proceeds in ice and is terminated by filtration, under vacuum, through Whatman GF/C glass fiber filters. The filters are washed twice with 5 ml of ice-cold 50 mM Tris.HCl (pH 7.4) and placed in scintillation vials. After drying at 50°-60° C. for 30 minutes, 10 ml of Beckman Ready-Solv™ HP (a high performance pre-mix scintillation cocktail, registered trademark of Beckman Instruments, Inc., Irvine, Calif. 92713) is added and the radioactivity is determined in a scintillation counter. Inhibition binding is calculated by the difference between total binding and binding in the presence of test compound, divided by the total binding, X 100. Active compounds of the invention will often inhibit binding in this assay by 10%, 20%, 30%, up to 50%, 75%, 95%, or greater compared to control assay results.

### EXAMPLE 16

### Demonstration of Sedative-Hypnotic Properties

The sedative-hypnotic properties of the novel compounds of the invention are established by their effect on the duration of ethanol induced narcosis in rats as a measurement of sedation. Groups of at least 8 rats are administered graded oral doses of the test compounds or vehicle 60 minutes prior to intraperitoneal treatment with 3.2 g/kg ethanol. Rats are then observed continuously for 180 minutes for the incidence and duration of ethanol induced narcosis. A rat is considered to exhibit narcosis if it remains in a supine position on a horizontal surface for at least 1 minute; the end of narcosis is defined as the rat spontaneously righting itself and remaining righted for at least 1 minute. The duration of narcosis is the total time the rat remained in a supine position. Test compounds are dissolved or suspended in a 2% aqueous starch suspension containing 5% polyethyleneglycol 400 and a drop of Tween®80; ethanol (95%) is adjusted to final concentration (V: V) with 0.85% saline. All treatments are administered in a constant volume of 5 ml/kg.

### EXAMPLE 17

### Demonstration of Skeletal Muscle Relaxant Activity

This test demonstrates efficacy of representative compounds of the invention to modulate the ability of rats to remain on an inclined screen. Groups of at least 6 rats are treated orally with graded doses of test compounds or vehicle and placed on a wire mesh screen (inclined at an angle of 60° from a horizontal level) 65 minutes later. The number of rats falling off the screen within 30 minutes is recorded. The ED₅₀ (dose necessary to cause 50% of the animals tested to fall off) is calculated according to the linear arc-sine transformation method of Finney, D. J. Statistical Methods in Biological Assay, 2nd Ed., Hafner, N.Y., 1964, pp. 454 ff. Compounds are dissolved or suspended in a 2% aqueous starch suspension containing 5% polyethylene glycol 400 and a drop of polysorbate 80, and are administered in a constant volume of 5 ml/kg.

### EXAMPLE 18

### Demonstration of Effects on Locomotor Activity

Groups of 6 rats are treated orally with vehicle (5 ml/kg) or graded doses of the test compounds. Sixty minutes later, individual rats are placed in Actophotometers and locomotor activity is measured for 5 minutes after a brief (30 sec.) habituation period. Motor Activity Counts (number of crossings of the photo cells) are recorded for each rat, and mean activity counts are calculated for each treatment group. The dose causing a 50% decrease in mean activity counts compared with the vehicle group (MDD₅₀) is calculated from a linear regression equation.

### EXAMPLE 19

### Protection During Ischemic Events

Corticostriatal coronal slices are prepared from 2- to 3-month-old Wistar rats (thickness, 270 to 300 μm). Slices are kept in artificial cerebrospinal fluid, composed as follows (in mmol/L): 126 NaCl, 2.5 KCl, 1.2 MgCl₂, 1.2 NaH2PO₄, 2.4 CaCl₂, 11 glucose, and 25 NaHCO₃. Artificial cerebrospinal fluid temperature is maintained at 34° C. and is gassed with O₂/CO₂ (95%/5%). In vitro ischemia is delivered by switching for 10 minutes to an artificial cerebrospinal fluid solution in which sucrose replaced glucose, gassed with 95% N₂ and 5% CO₂. Ischemic and drug-containing solutions enter the recording chamber no later than 30 seconds after a 3-way tap is turned. Compositions of formula I (for example containing a monochloro or monobromo derivative) are applied by dissolving them to the desired final concentration in saline solution.

Electrodes for extracellular recordings (15 to 20 MΩ) are filled with 2 mol/L NaCl. An Axoclamp 2B amplifier (Axon Instruments) is used for extracellular recordings. The field potential amplitude is defined as the average of the amplitude from the peak of the early positivity to the peak negativity and the amplitude from the peak negativity to peak late positivity. Quantitative data on modifications induced by ischemia are expressed as a percentage of the control values, the latter representing the mean of responses recorded during a stable period (15 to 20 minutes) before the ischemic phase. Tracings are displayed on a digital oscilloscope (Classic 6000, Gould) and digitally stored. Under control conditions, the ischemic period (10 minutes) produced an irreversible loss of field potential. Pre-incubation with a composition of formula I will partially or fully reverse the effect of the ischemic event.

### EXAMPLE 20

### The Maximal Electroshock Seizure (MES) or Maximal Seizure Pattern Test

The MES is an experimental model predictive of drug activity for controlling generalized tonic-clonic seizures and preventing seizure spread. An advantage of this model is that the behavioral and electrographic seizures are consistent with those observed in humans.

In the MES test, the animal receives an electrical stimulus, 0.2 seconds in duration, via corneal electrodes primed with an electrolyte solution containing an anesthetic agent. The 0.2 second stimulation is generated with 150 mA in rats and 50 mA in mice at 60 Hz. Rats, weighing between 105 g and 130 g, and mice, weighing between 18 g and 25.5 g, receive an electrical stimulus 15 minutes, 30 minutes, 1 hour, 2 hours, and 4 hours after administration of the test compound. In rats, the compound is administered orally, while mice receive the agent via intraperitoneal injection. The test endpoint, electrogenic seizure, is manifested as hindlimb tonic extension. Inhibition of hindlimb tonic extension indicates that the test compound is able to inhibit MES-induced seizure spread and therefore is likely to have effective antiseizure activity.

Although the foregoing invention has been described in detail by way of example for purposes of clarity of understanding, it will be apparent to the artisan that certain changes and modifications may be practiced within the scope of the appended claims which are presented by way of illustration not limitation. In this context it will be understood that this invention is not limited to the particular formulations, process steps, and materials disclosed herein as such formulations, process steps, and materials may vary somewhat. It will also be understood that the terminology employed herein is used for the purpose of describing particular embodiments only, and is not intended to be limiting since the scope of the present invention will be limited only by the appended claims and equivalents thereof. It is further noted that various publications and other reference information have been cited within the foregoing disclosure for economy of description. Each of these references are incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

P S Albright, W M Burnham. Development of a new pharmacological seizure model: effects of anticonvulsants on cortical- and amygdala-kindled seizures in the rat. Epilepsia. 1980 December; 21(6):681-9.
E A Barnard, P Skolnick, R W Olsen, H Mohler, W Sieghart, G Biggio, C Braestrup, A. N. Bateson, S. Z. Langer. International Union of Pharmacology. XV. Subtypes of gamma-aminobutyric acidA receptors: classification on the basis of subunit structure and receptor function. Pharmacol Rev. 1998 June; 50(2):291-313.
N W Dunham, T S Miya. A note on a simple apparatus for detecting neurological deficit in rats and mice. J Am Pharm Assoc Am Pharm Assoc (Baltim). 1957 March; 46(3):208-9.
Paul R. Fleming, K. Barry Sharpless. Selective transformations of threo-2,3-dihydroxy esters. J. Org. Chem.; 1991; 56(8); 2869-2875.
Albert D. Fraser, Stanley J. Clark, Herbert H. Wotiz. Demethylation of labile aryl ethers. J. Org. Chem.; 1976; 41(1); 170-171.
R. T. Hill and D. H. Tedeschi, “Animal Testing and Screening Procedures in Evaluating Psychotropic Drugs” in “An Introduction to Psychopharmacology”, Eds. R. R. Rech and K. E. Moore, Raven Press, New York, pp 237-288 (1971).
S. R. Kay, A Fiszbein, L A Opler. The positive and negative syndrome scale (PANSS) for schizophrenia. Schizophr Bull. 1987; 13(2):261-76.
Joseph J. Kopcho, James C. Schaeffer. Selective O-demethylation of 7-α-aminomethyl)6,14-endo-ethenotetrahydrothebaine J. Org. Chem.; 1986; 51(9); 1620-1622.
R L Krall R L, J K Penry, B G White, H J Kupferberg, E A Swinyard. Antiepileptic drug development: II. Anticonvulsant drug screening. Epilepsia. 1978 August; 19(4):409-28.
Skolnick, et al., J. Pharmacol. Exper. Ther. 283:488493, 1997, and Liu, et al., J. Med. Chem. 39:1928-1934,1996.
E. Harold Vickery, Leon F. Pahler, Edmund J. Eisenbraun. Selective O-demethylation of catechol ethers. Comparison of boron tribromide and iodotrimethylsilane. J. Org. Chem.; 1979; 44(24); 4444-4446.
Laurie W. Wisden, H. Monyer, P H Seeburg. The distribution of 13 GABA_{A} receptor subunit mRNAs in the rat brain. I. Telencephalon, diencephalon, mesencephalon. J. Neurosci. 1992 March; 12(3):1040-62.
P. Wlaz, W. Loscher. Weak anticonvulsant effects of two novel glycineB receptor antagonists in the amygdala-kindling model in rats. Eur J. Pharmacol. 1998 Jan 19;342(1):39-46.
J. R. Vogel, B. Beer and D. E. Clody, “A Simple and Reliable Conflict Procedure for Testing Anti-Anxiety Agents”, Psychopharmacologia, 21, 1-7 (1971).

1. A compound of the formula I: 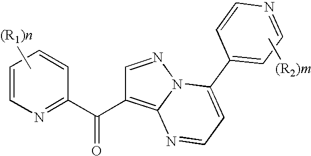
wherein each R₁ is independently selected from a halogen, hydroxy, alkyl, alkoxy, nitro, amino, trifluoromethyl, cycloalkyl, (cycloalkyl)alkyl, alkanoyl, alkanoyloxy, aryl, aroyl, aralkyl, nitrile, pyrrolidine-1-yl, morpholino, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, alkanoylamino, carbamoyl, carbamyl, carbonylamino, alkylsulfonylamino, or heterocyclo group; and
wherein each R₂ is independently selected from a halogen, hydroxy, alkyl, alkoxy, nitro, amino, trifluoromethyl, cycloalkyl, (cycloalkyl)alkyl, alkanoyl, alkanoyloxy, aryl, aroyl, aralkyl, nitrile, pyrrolidine-1-yl, morpholino, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, alkanoylamino, carbamoyl, carbamyl, carbonylamino, alkylsulfonylamino, or heterocyclo group.

2. The compound of claim 1, wherein n is 1.

3. The compound of claim 1, wherein n is between 1 and 4.

4. The compound of claim 1, wherein m is 1.

5. The compound of claim 1, wherein m is between 1 and 4.

6. The compound of claim 1, wherein two R₁ or R₂ groups, which can be identical or different, are fused to form a five-membered ring.

7. The compound of claim 1, wherein two R₁ or R₂ groups, which can be identical or different, are fused to form a six-membered ring.

8. The compound of claim 1, wherein each R₁ or R₂, which can be identical or different, is an alkyl substituted with from one to three substituents selected from the group consisting of halogen, hydroxy and amino.

9. The compound of claim 1, wherein each R₁ or R₂, which can be identical or different, is selected from the group consisting of methyl, methoxy, cyclopropyl, acetyl and acetoxy groups.

10. The compound of claim 1, wherein each R₁ or R₂, which can be identical or different, is an aryl, aroyl, arylalkyl or heterocyclo group.

11. The compound of claim 10, wherein said aryl, aroyl, aralkyl or heterocyclo groups are substituted with one to four substituents selected from the group consisting of alkyl, substituted alkyl, halogen, trifluoromethyl, trifluoromethoxy, hydroxy, alkoxy, cycloalkyloxy, alkanoyl, alkanoyloxy, amino, alkylamino, dialkylamino, nitro, cyano, carboxy, carboxyalkyl, carbamyl, carbamoyl and aryloxy.

12. The compound of claim 10, wherein said aryl group is selected from the group consisting of substituted or unsubstituted phenyl, naphthyl, biphenyl and diphenyl groups.

13. The compound of claim 12, wherein the substituted phenyl, naphthyl, biphenyl or diphenyl groups are substituted with one to four substituents selected from the group consisting of alkyl, substituted alkyl, halogen, trifluoromethyl, trifluoromethoxy, hydroxy, alkoxy, cycloalkyloxy, alkanoyl, alkanoyloxy, amino, alkylamino, dialkylamino, nitro, cyano, carboxy, carboxyalkyl, carbamyl, carbamoyl and aryloxy.

14. The compound of claim 10, wherein said aroyl group is selected from the group consisting of substituted or unsubstituted benzoyl and naphthoyl groups.

15. The compound of claim 14, wherein said substituted benzoyl and naphthoyl groups are substituted with one to four substituents selected from the group consisting of alkyl; substituted alkyl, halogen, trifluoromethyl, trifluoromethoxy, hydroxy, alkoxy, cycloalkyloxy, alkanoyl, alkanoyloxy, amino, alkylamino, dialkylamino, nitro, cyano, carboxy, carboxyalkyl, carbamyl, carbamoyl and aryloxy.

16. The compound of claim 10, wherein said arylalkyl group is substituted or unsubstituted benzyl.

17. The compound of claim 16, wherein said substituted benzyl is substituted with one to four substituents selected from the group consisting of alkyl, substituted alkyl, halogen, trifluoromethyl, trifluoromethoxy, hydroxy, alkoxy, cycloalkyloxy, alkanoyl, alkanoyloxy, amino, alkylamino, dialkylamino, nitro, cyano, carboxy, carboxyalkyl, carbamyl, carbamoyl and aryloxy.

18. The compound of claim 10, wherein said heterocyclo group is monocyclic.

19. The compound of claim 10, wherein said heterocyclo group is bicyclic.

20. The compound of claim 10, wherein said heterocyclo group is selected from the group consisting of pyrrolidinyl, pyrrolyl, indolyl, pyrazolyl, imidazolyl, oxazolyl, isoxazolyl, thiazolyl, furyl, tetrahydrofuryl, thienyl, piperidinyl, piperazinyl, azepinyl, pyrimidinyl, pyridazinyl, tetrahydropyranyl, morpholinyl, dioxanyl, triazinyl, triazolyl, benzothiazolyl, benzoxazolyl, benzothienyl, quinolinyl, tetrahydroisoquinolinyl, benzimidazolyl, benzofuryl, indazolyl, benzisothiazolyl, isoindolinyl and tetrahydroquinolinyl.

21. The compound of claim 20, wherein said heterocyclo group is substituted with one to four substituents selected from the group consisting of alkyl, substituted alkyl, halogen, trifluoromethyl, trifluoromethoxy, hydroxy, alkoxy, cycloalkyloxy, alkanoyl, alkanoyloxy, amino, alkylamino, dialkylamino, nitro, cyano, carboxy, carboxyalkyl, carbamyl, carbamoyl and aryloxy.

22. The compound of claim 1, wherein said halogen substituent is chloro or bromo, and wherein two R₁ or R₂ groups, each of which can be identical or different, together with the ring carbons to which they are attached, form a quinolin-4-yl group.

23. The compound of claim 1, wherein two R₁ groups, together with the ring carbons to which they are attached, form an isoquinolinyl moiety.

24. The compound of claim 1, wherein the compound is (6-methyl-2-pyridinyl)-[7-(2-chloropyridin-4-yl)pyrazolo[1,5-a]pyrimidin-3-yl]methanone; (6-methoxy-2-pyridinyl)-[7-(2-chloropyridin-4-yl)pyrazolo[1,5-a]pyrimidin-3-yl]methanone; (6-methoxy-2-pyridinyl)-[7-(2-bromopyridin-4-yl)pyrazolo[1,5-a]pyrimidin-3-yl]methanone; or (6-methyl-2-pyridinyl)-[7-(2-bromopyridin-4-yl)pyrazolo[1,5-a]pyrimidin-3-yl]methanone.

25. A method for the treating or preventing a neurological or psychiatric disorder mediated by a defect or disturbance in GABA or GABA_{A} receptor physiology in a mammalian subject comprising, administering to said subject a GABA- or GABA_{A} receptor-modulating effective amount of a compound of claim 1.

26. The method of claim 25, further comprising administering a second GABA- or GABA_{A} receptor-modulating agent, wherein the second GABA- or GABA_{A} receptor-modulating agent is an anxiolytic, antidepressant, anticonvulsant, nootropic, anesthetic, hypnotic, or muscle relaxant agent.

27. The method of claim 26, wherein the second GABA- or GABA_{A} receptor-modulating agent is administered to said subject in a combined formula with the compound of claim 1.

28. The method of claim 26, wherein the second GABA- or GABA_{A} receptor-modulating agent is administered to said subject in a coordinate administration protocol, simultaneously with, prior to, or after administration of said compound of claim 1 to the subject.

29. The method of claim 25, wherein the disorder is stroke, head trauma, epilepsy, pain, migraine, mood disorders, anxiety, post traumatic stress disorder, obsessive compulsive disorders, mania, bipolar disorders, schizophrenia, seizures, convulsions, tinnitus, neurodegenerative disorder, Alzheimer's disease, amyotrophic lateral sclerosis, Parkinson's disease, Huntington's chorea, depression, bipolar disorders, mania, trigeminal neuralgia, neuralgia, neuropathic pain, hypertension, cerebral ischemia, cardiac arrhythmia, myotonia, substance abuse, myoclonus, essential tremor, dyskinesia, movement disorders, neonatal cerebral hemorrhage, or spasticity.

30. The method of claim 25, wherein the disorder is anxiety.

31. The method of claim 25, wherein the disorder is epilepsy.

32. The method of claim 25, wherein the effective amount is between about 1 mg to about 600 mg per day.

33. The method of claim 25, wherein the effective amount is between about 50 mg to about 300 mg per day.

34. A composition for eliciting a therapeutic response mediated by modulation of GABA or GABA_{A} receptor physiology in a mammalian subject comprising, administering to said subject an effective amount of a compound of formula I or a pharmaceutically acceptable salt, solvate, hydrate, polymorph, or prodrug thereof.
